(19) United States
(12) Patent Application Publication
(10) Pub. No.: US 2010/0316575 A1
(43) Pub. Date: Dec. 16, 2010

(54) MODULATING BODY WEIGHT

(76) Inventors: Leonid V. Zingman, Iowa City, IA (US); Andre Terzic, Rochester, MN (US); Alexey E. Alekseev, Rochester, MN (US); Satsuki Yamada, Rochester, MN (US); Nicolas J. Niederlander, Rochester, MN (US); Santiago Reyes Ramirez, Rochester, MN (US)

Correspondence Address: FISH & RICHARDSON P.C. (TC), PO BOX 1022, MINNEAPOLIS, MN, 55440-1022, US

(21) Appl. No.: 12/865,846

(22) PCT Filed: Jan. 28, 2009
(86) PCT No.: PCT/US09/32184

§ 371 Date: Sep. 2, 2010

Related U.S. Application Data
(60) Provisional application No. 61/025,594, filed on Feb. 1, 2008.

(51) Int. Cl. A61K 31/23; A61K 31/64; A61K 49/00; A61P 3/04

(52) U.S. Cl. 424/9.2; 514/584; 514/552

(57) ABSTRACT

This document provides methods and materials related to modulating body weight. For example, methods and materials related to treating obesity, reducing body weight, preventing body weight gain, increasing body weight, and identifying agents having the ability to treat obesity and reduce body weight are provided.

### CROSS-REFERENCE TO RELATED APPLICATIONS

This application claims the benefit of U.S. Provisional Application Ser. No. 61/025,594, filed Feb. 1, 2008.

### STATEMENT AS TO FEDERALLY SPONSORED RESEARCH

This invention was made with government support under grant HL064822 awarded by the National Institutes of Heart, Lung, and Blood Institute. The government has certain rights in the invention.

### BACKGROUND

1. Technical Field

This document relates to methods and materials involved in modulating body weight. For example, this document relates to methods and materials involved in modulating body weight using potassium channel agonists or antagonists.

2. Background Information

The balance between energy availability and consumption defines body weight. Sur-alimentation and sedentary lifestyle have caused an enormous growth in the obese population worldwide. The epidemic in obesity has produced a surge in the prevalence of systemic debilitating co-morbidities, including diabetes and heart failure, with an immense impact on global health.

Metabolic homeostasis relies on feedback loops between the regulatory neuroendocrine system, and peripheral energy-consuming and energy-storing tissues. Due to limited food availability in the natural habitat, evolutionary pressure favors systems that defend against energy and body weight loss. While naturally protective, energy-saving systems, under conditions of excessive energy supply, promote obesity.

### SUMMARY

This document provides methods and materials related to modulating body weight. For example, this document provides methods and materials related to treating obesity, reducing body weight, preventing body weight gain, increasing body weight, identifying agents having the ability to treat obesity and reduce body weight, and identifying agents having the ability to increase body weight.

In general, one aspect of this document features a method for modulating body weight in a mammal. The method comprises administering, to the mammal, a composition comprising a potassium channel agonist, a potassium channel antagonist, an agent having the ability to increase potassium channel expression in the mammal, or an agent having the ability to decrease potassium channel expression in the mammal. The mammal can be a human. The mammal can be overweight, and the composition can comprise the potassium channel antagonist. The composition can be administered to the mammal under conditions wherein the body weight of the mammal decreases. The potassium channel antagonist can be HMR 1883, HMR 1098, 5-hydroxydecanoate, 1-[5-[2-(5-chloro-o-anisamido)ethyl]-2-methoxyphenyl]sulfonyl-3-methylthiourea, or sulfonylurea drugs. The mammal can be overweight, and the composition can comprise the agent having the ability to decrease potassium channel expression in the mammal.

In another aspect, this document features a method for identifying a treatment agent for modulating body weight. The method comprises (a) determining whether or not a test agent modulates potassium channel activity, wherein modulation of the potassium channel activity indicates that the test agent is a candidate agent, and (b) administering the candidate agent to a mammal to determine whether or not the candidate agent modulates the weight of the mammal or modulates the rate of weight gain in the mammal, wherein a modulation of the weight or the rate of weight gain indicates that the candidate agent is the treatment agent. The step (a) can comprise using an in vitro potassium channel activity assay. The mammal can be a mouse.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. Individually housed and at libitum fed mice lacking functional K_{ATP} channels, through knockout of KCNJ11-encoded Kir6.2 pore (Kir6.2-KO), demonstrated significantly smaller body weights than age and gender-matched wild-type (WT) counterparts, as early as at 5-months of age and throughout the 50 weeks of follow-up (FIGS. 1A and 1B). The reduced body weight was not a consequence of altered growth, as through the observation period Kir6.2-KO displayed similar heights as the WT, and reached 12.2±0.2 cm (n=10 Kir6.2-KO) versus 12.4±0.1 cm (n=10 WT) by 12 months of age (FIG. 1C and FIG. 1D). As a result, the body mass index and waist-to-height index of Kir6.2-KO were both significantly reduced compared to the WT, as exemplified at 12 months of age (FIGS. 1C and 1D). Whole body nuclear magnetic resonance scans indicated that the reduced body weight of 1-year old Kir6.2-KO was associated with depletion of subcutaneous and abdominal fat depots compared to matched WT controls (FIG. 1E-G). Abbreviations: D; diaphragm, IF; interstitial fat, K; kidney, PF; retroperitoneal fat, SC; spinal cord, SF; subcutaneous fat.

FIG. 2. Macroscopic differences in body weight and fat distribution were preceded by a reduction in white adipose tissue size in Kir6.2-KO already at 3-months of age, revealed by histological examination (FIGS. 2A and 2B). Maturation in the WT was associated with a doubling in the average adipocyte area within 12 months, an enlargement not observed in the Kir6.2-KO (FIGS. 2C and 2D). FIG. 3. Continuous monitoring by telemetry detected a similar routine activity in sedentary WT (n=10) and Kir6.2-KO (n=10) mice throughout the day-night cycle (FIG. 3A). In fact, both WT and Kir6.2-KO spent equal time either without (FIG. 3B) or with (FIG. 3C) movement. Change in physical activity can thus be excluded as a cause of K_{ATP} channel-dependent control of body weight. Weight maintenance, in the setting of equivalent activity, is governed by food intake and/or energy consumption, the later including energy-dependent support of posture maintenance and non-exercise routine, as well as energy expenditure for basic bodily functions. At similar serum levels of the satiety hormone leptin (FIG. 3H), disparity in energy supply as grounds for distinct trends in body weight maintenance was excluded as Kir6.2-KO demonstrated a higher food intake than WT counterparts (FIG. 3D). Moreover, although Kir6.2 has been implicated in the regulation of glucose homeostasis with marginal impairment in glucose tolerance, no significant difference in energy equivalents at organ levels could be detected in the presence versus absence of functional K_{ATP} channels (FIG. 3E-I). Specifically, similar non-fasting blood glucose concentrations, comparable serum levels of glucose-regulating hormones insulin, amylin and resistin (FIG. 3D), as well as equivalent glucose/insulin ratio despite lower serum glucagon levels (FIG. 3D) and a higher insulin sensitivity were detected in the Kir6.2-KO compared to WT (FIG. 3E-H).

FIG. 4. Under sedentary conditions, during continuous 24-h monitoring, the Kir6.2-KO cohort demonstrated a higher rate of oxygen consumption throughout the 12-h light cycles compared to age-, sex-, weight- and body fat distribution-matched WT (FIG. 4A). Concomitant measurement of the rate of O₂ consumption and CO₂ production revealed a higher rate of caloric output normalized to whole body weight, reflecting a higher energy expenditure (EE=3.815·\dot{V} O₂+1.232·\dot{V} CO₂), during both night and day, in Kir6.2-KO (n=11) compared to the WT (n=11; FIG. 4B). To evaluate the K_{ATP} channel contribution on the energetic cost of physical activity indirect calorimetry was here performed in WT and Kir6.2-KO mice during exercise treadmill test. At an inclination of 5°, treadmill speed was individually set according to body weight to achieve a workload of 2 mJ/s (2 mW). With initiation of exercise test and despite prior conditioning, all animals experienced a stress-induced transient elevation of oxygen consumption ultimately reaching steady levels when oxygen consumption matched workload (FIG. 4C). At steady-state, the energetic cost of 2 mW workload was significantly higher in Kir6.2-KO compared to WT expressed either as rate of oxygen consumption or energy expenditure (FIGS. 4C and 4D). Maximal oxygen consumption, reflecting total oxygen phosphorylation potential, was equivalent in WT and Kir6.2-KO (FIGS. 4C and 4D).

FIG. 5. Telemetry recording of cardiac activity defined that both WT and Kir6.2-KO mice display a heart rate profile with 400 to 800 beats per minute (FIG. 5A), yet when isolated hearts were retrogradly perfused and paced in this physiological range defective rate-dependent modulation of action potential duration was revealed in the absence of K_{ATP} channels (FIG. 5B). While with increase in heart rate the WT (n=5) prominently shortened monophasic action potential duration at 90% repolarization (APD₉₀), Kir6.2-KO hearts (n=8) demonstrated a significantly reduced action potential shortening especially at high heart rates (P<0.05; FIG. 5B). The K_{ATP} channel-dependent component in rate-dependent action potential shortening was verified in WT hearts following K_{ATP}-channel blockade with the sulfonylurea blocker glyburide (FIG. 5B). The inability to limit action potential duration translated into significantly higher levels of oxygen consumption and glycogen storage depletion in the absence of functional K_{ATP} channels (FIG. 5C). Maximal oxygen consumption (V O₂ₘₐₓ), corresponding to the highest tolerated workload, reflects the maximal rate of energy production and the ability of the cardiovascular system to supply oxygen. Kir6.2-KO and WT mice achieved the same level of V O₂ₘₐₓ reflecting their equivalent oxidative phosphorylation potential. However, Kir6.2-KO animals reached V O₂ₘₐₓ level already at 30-35 mW (n=7)), a value of workload power significantly lower than the 40-45 mW when WT mice reached this level (n=7; FIG. 5D). Analysis of the rate of oxygen consumption during maximally tolerated treadmill workload revealed a significantly slower rate and larger oxygen deficit in Kir6.2-KO mice compared to WT (FIG. 5D). On cessation of the exercise workload regimen, Kir6.2-KO mice displayed a collapse in oxygen consumption (FIG. 5D). This was associated with high lactate levels in the blood (FIG. 5E), indicating compromise of the cardiovascular system.

FIG. 6. Microarray chips revealed 97 genes which displayed 50% or more change in levels in Kir6.2-KO compared to WT (FIG. 6A and FIG. 6B). Data were verified by QT-PCR and immunocytochemistry for Phkb, Stard5, Ankrd23, Nppa and Thrsp (FIG. 6C-6E).

FIG. 7. Obesity resistance was demonstrated on 130-days of a high fat diet regimen in Kir6.2-KO versus WT despite higher food consumption (FIG. 7A-7C).

FIG. 8. Tabular representation of the gene list pertinent to the comparison of WT versus Kir6.2-KO genome expression analysis (see FIG. 6).

### DETAILED DESCRIPTION

This document provides methods and materials related to modulating body weight. For example, this document provides methods and materials related to treating obesity, reducing body weight, preventing body weight gain, increasing body weight, identifying agents having the ability to treat obesity and reduce body weight, and identifying agents having the ability to increase body weight.

As described herein, modulation of potassium channel function and coupling with the cellular metabolic state can be used to control body weight. For example, reduction of potassium channel activity can be used to treat obesity in a mammal, reduce a mammal's body weight, or prevent body weight gain in a mammal. The mammal can be any type of mammal including, without limitation, a mouse, rat, dog, cat, horse, sheep, goat, cow, pig, monkey, or human.

A potassium channel antagonist can be any agent that inhibits or reduces the activity of a potassium channel. A potassium channel agonist can be any agent that activates or increases the activity of a potassium channel. The potassium channel can be a human K_{ATP} channel. Examples of potassium channel antagonists include, without limitation, HMR 1883, HMR 1098, 5-hydroxydecanoate, 1-[5-[2-(5-chloro-o-anisamido)ethyl]-2-methoxyphenyl]sulfonyl-3-methylthiourea, and sulfonylurea drugs. Examples of potassium channel agonists include, without limitation, Nicorandil, Minoxidil Sulphate, Diazoxide, Pinacidil, Leveromakalim, and Cromakalim.

In some cases, an agent having the ability reduce potassium channel expression can be used in place of a potassium channels antagonist or in combination with a potassium channels antagonist. Examples of agents having the ability reduce potassium channel expression include, without limitation, siRNA molecules, antisense oligonucleotides, and peptide nucleic acids designed to reduce potassium channel expression.

In some cases, a potassium channel antagonist can be an antibody having the ability to bind to a potassium channel. Such an antibody can be, without limitation, a polyclonal, monoclonal, human, humanized, chimeric, or single-chain antibody, or an antibody fragment having binding activity, such as a Fab fragment, F(ab′) fragment, Fd fragment, fragment produced by a Fab expression library, fragment comprising a VL or VH domain, or epitope binding fragment of any of the above. An antibody can be of any type (e.g., IgG, IgM, IgD, IgA or IgY), class (e.g., IgG1, IgG4, or IgA2), or subclass. In addition, an antibody can be from any animal including birds and mammals. For example, an antibody can be a human, rabbit, sheep, or goat antibody. An antibody can be naturally occurring, recombinant, or synthetic. Antibodies can be generated and purified using any suitable methods known in the art. For example, monoclonal antibodies can be prepared using hybridoma, recombinant, or phage display technology, or a combination of such techniques. In some cases, antibody fragments can be produced synthetically or recombinantly from a gene encoding the partial antibody sequence. An anti-potassium channel antibody can bind to a potassium channel polypeptide at an affinity of at least 10⁴ mol⁻¹ (e.g., at least 10⁵, 10⁶, 10⁷, 10⁸, 10⁹, 10¹⁰, 10¹¹, or 10¹² mol⁻¹).

An anti-potassium channel antibody provided herein can be prepared using any appropriate method. For example, any substantially pure potassium channel polypeptide, or fragment thereof (e.g., a truncated potassium channel polypeptide), can be used as an immunogen to elicit an immune response in an animal such that specific antibodies are produced. Thus, a human potassium channel polypeptide or a fragment thereof can be used as an immunizing antigen. In addition, the immunogen used to immunize an animal can be chemically synthesized or derived from translated cDNA. Further, the immunogen can be conjugated to a carrier polypeptide, if desired. Commonly used carriers that are chemically coupled to an immunizing polypeptide include, without limitation, keyhole limpet hemocyanin (KLH), thyroglobulin, bovine serum albumin (BSA), and tetanus toxoid.

The preparation of polyclonal antibodies is well-known to those skilled in the art. See, e.g., Green et al., Production of Polyclonal Antisera, in IMMUNOCHEMICAL PROTOCOLS (Manson, ed.), pages 1-5 (Humana Press 1992) and Coligan et al., Production of Polyclonal Antisera in Rabbits, Rats, Mice and Hamsters, in CURRENT PROTOCOLS IN IMMUNOLOGY, section 2.4.1 (1992). In addition, those of skill in the art will know of various techniques common in the immunology arts for purification and concentration of polyclonal antibodies, as well as monoclonal antibodies (Coligan, et al., Unit 9, CURRENT PROTOCOLS IN IMMUNOLOGY, Wiley Interscience, 1994).

The preparation of monoclonal antibodies also is well-known to those skilled in the art. See, e.g., Kohler & Milstein, Nature 256:495 (1975); Coligan et al., sections 2.5.1 2.6.7; and Harlow et al., ANTIBODIES: A LABORATORY MANUAL, page 726 (Cold Spring Harbor Pub. 1988). Briefly, monoclonal antibodies can be obtained by injecting mice with a composition comprising an antigen, verifying the presence of antibody production by analyzing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well established techniques. Such isolation techniques include affinity chromatography with Protein A Sepharose, size exclusion chromatography, and ion exchange chromatography. See, e.g., Coligan et al., sections 2.7.1 2.7.12 and sections 2.9.1 2.9.3; Barnes et al., Purification of Immunoglobulin G (IgG), in METHODS IN MOLECULAR BIOLOGY, Vol. 10, pages 79-104 (Humana Press 1992).

Once hybridoma clones that produce antibodies to an antigen of interest (e.g., a potassium channel polypeptide) have been selected, further selection can be performed for clones that produce antibodies having a particular specificity. For example, clones can be selected that produce antibodies that preferentially bind to a potassium channel polypeptide and inhibit potassium channel polypeptide activity.

The antibodies provided herein can be substantially pure. The term “substantially pure” as used herein with reference to an antibody means the antibody is substantially free of other polypeptides, lipids, carbohydrates, and nucleic acid with which it is naturally associated in nature. Thus, a substantially pure antibody is any antibody that is removed from its natural environment and is at least 60 percent pure. A substantially pure antibody can be at least about 65, 70, 75, 80, 85, 90, 95, or 99 percent pure.

In general, excessive overweight and obesity can be treated by administering a potassium channel antagonist or an agent having the ability to reduce potassium channel expression to a mammal in need of weight reduction. It will be appreciated that a single potassium channel antagonist or agent having the ability to reduce potassium channel expression can be used to reduce body weight or a combination of such antagonists and/or agents (e.g., two, three, four, five, or more potassium channel antagonists) can be used to reduce body weight.

Any appropriate method can be used to administer a composition provided herein to a mammal. For example, a potassium channel antagonist can be administered orally or via injection (e.g., subcutaneous injection, intramuscular injection, intravenous injection, or intrathecal injection). In some cases, a combination of potassium channel antagonists can be administered by different routes. For example, one potassium channel antagonist can be administered orally and a second potassium channel antagonist can be administered via injection.

Before administering a composition provided herein (e.g., a composition containing one or more potassium channel antagonists) to a mammal, the mammal can be assessed to determine the mammal's body weight. After determining the mammal's body weight, the mammal can be administered a composition to increase body weight (e.g., a composition containing one ore more potassium channel agonists) or a composition to decrease body weight (e.g., a composition containing one or more potassium channel antagonists).

A composition provided herein can be administered to a mammal in any amount, at any frequency, and for any duration effective to achieve a desired outcome (e.g., to increase or decrease body weight). In some cases, a composition provided herein can be administered to a mammal to increase or decrease body weight 1, 2, 3, 4, 5, 10, 15, 20, 25, 30, 35, 40, 45, 50, or more percent.

An effective amount of a composition provided herein can be any amount that modulates body weight (e.g., increase or decreases body weight) without producing significant toxicity to the mammal. For example, an effective amount of a potassium channel antagonist can be from about 0.05 mg/kg to about 100 mg/kg (e.g., from about 0.1 mg/kg to about 50 mg/kg, from about 0.2 mg/kg to about 25 mg/kg, or from about 0.5 mg/kg to about 10 mg/kg). Typically, an effective amount of a potassium channel antagonist, such as the sulfonylurea glyburide, can reach at tissue level the concentration in the range of 5 μM to 10 μM. If a particular mammal fails to respond to a particular amount, then the amount can be increased by, for example, two fold. After receiving this higher concentration, the mammal can be monitored for both responsiveness to the treatment and toxicity symptoms, and adjustments made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the weight condition may require an increase or decrease in the actual effective amount administered.

The frequency of administration can be any frequency that modulates body weight without producing significant toxicity to the mammal. For example, the frequency of administration can be from about once a week to about three times a day, or from about twice a month to about six times a day, or from about twice a week to about once a day. The frequency of administration can remain constant or can be variable during the duration of treatment. A course of treatment with a composition provided herein can include rest periods. For example, a potassium channel antagonist can be administered daily over a two week period followed by a two week rest period, and such a regimen can be repeated multiple times. As with the effective amount, various factors can influence the actual frequency of administration used for a particular application. For example, the effective amount, duration of treatment, use of multiple treatment agents, route of administration, and severity of the weight condition may require an increase or decrease in administration frequency.

An effective duration for administering a composition provided herein can be any duration that modulates weight without producing significant toxicity to the mammal. Thus, the effective duration can vary from several days to several weeks, months, or years. In general, the effective duration for the treatment of obesity can range in duration from several months to several years. In some cases, an effective duration can be for as long as an individual mammal is alive. Multiple factors can influence the actual effective duration used for a particular treatment. For example, an effective duration can vary with the frequency of administration, effective amount, use of multiple treatment agents, route of administration, and severity of the weight condition.

A composition containing a potassium channel antagonist, a potassium channel agonist, or an agent that increases or decreases expression of a potassium channel can be in any appropriate form. For example, a composition provided herein can be in the form of a solution or powder with or without a diluent to make an injectable suspension. A composition also can contain additional ingredients including, without limitation, pharmaceutically acceptable vehicles. A pharmaceutically acceptable vehicle can be, for example, saline, water, lactic acid, and mannitol.

Suitable formulations for oral administration can include tablets or capsules prepared by conventional means with pharmaceutically acceptable excipients such as binding agents (e.g., pregelatinized maize starch, polyvinylpyrrolidone or hydroxypropyl methylcellulose), fillers (e.g., lactose, microcrystalline cellulose or calcium hydrogen phosphate), lubricants (e.g., magnesium stearate, talc or silica), disintegrants (e.g., potato starch or sodium starch glycolate), or wetting agents (e.g., sodium lauryl sulfate). Tablets can be coated by methods known in the art. Preparations for oral administration can also be formulated to give controlled release of the agent.

After administering a composition provided herein to a mammal, the mammal can be monitored to determine whether or not body weight was modulated. For example, a mammal can be assessed after treatment to determine whether or not body weight was reduced.

This document also provides methods and materials related to treating mammals (e.g., humans) likely to develop over or under weight conditions (e.g., mammals having an elevated risk of developing diet-induced obesity). A mammal can be identified as having or being likely to develop a weight condition using standard clinical techniques. For example, analysis of a human's family history or eating habits can be used to determine whether or not the human is likely to develop an obesity condition. As described herein, a mammal identified as having or being susceptible to developing a weight condition can be treated by administering a composition provided herein.

This document also provides methods and materials for identifying agents that can be used to modulate body weight in a mammal. Such agents can be identified by screening candidate agents (e.g., from synthetic compound libraries and/or natural product libraries) for potassium channel agonist or antagonist activity. Candidate agents can be obtained from any commercial source and can be chemically synthesized using methods that are known to those of skill in the art. Candidate agents can be screened and characterized using in vitro cell-based assays, cell free assays, and/or in vivo animal models. For example, a potassium channel activity assay such as the patch-clamp electrophysiological method of monitoring K^{ATP} channel activity can be used to identify agents that can be used to modulate body weight in a mammal. Agents can be confirmed as having the ability to modulate body weight by administering the agents to animal models or humans.

The invention will be further described in the following examples, which do not limit the scope of the invention described in the claims.

### EXAMPLES

### Example 1

### ATP-Sensitive K+ Channel Axis Governs Body Energy Expenditure Through Exercise and Non-Exercise Activity Thermogenesis

K_{ATP} Channel Deletion Causes Reduced Body Weight with Fat Depletion

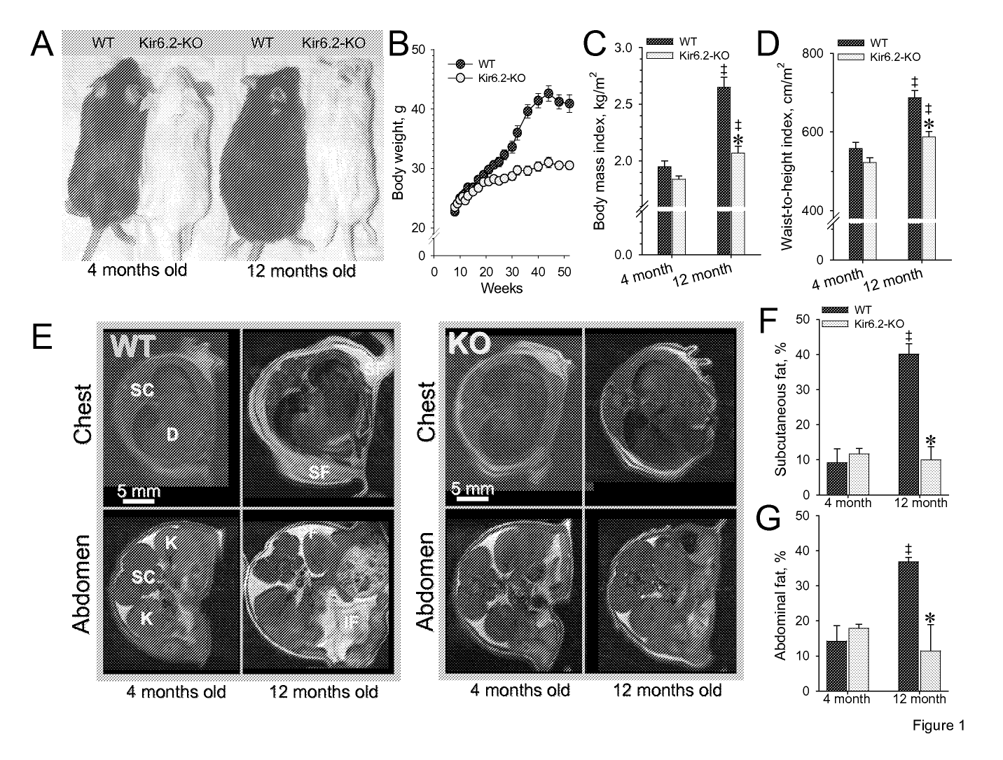

Body weight is the integrative index of the balance between energy availability and consumption. Individually housed and at libitum fed mice lacking functional K_{ATP} channels, through knockout of KCNJ11-encoded Kir6.2 pore (Kir6.2-KO), exhibited significantly smaller body weights than age and gender-matched wild-type (WT) counterparts, as early as at 5-months of age and throughout the 50 weeks of follow-up (FIGS. 1A and 1B). The reduced body weight was not a consequence of altered growth, as through the observation period Kir6.2-KO mice exhibited similar heights as WT mice, and reached 12.2±0.2 cm (n=10 Kir6.2-KO mice) versus 12.4±0.1 cm (n=10 WT mice) by 12 months of age. As a result, the body mass index and waist-to-height index of Kir6.2-KO mice were both significantly reduced compared to WT mice, as exemplified at 12 months of age (FIGS. 1C and 1D). Whole body nuclear magnetic resonance scans indicated that the reduced body weight of 1-year old Kir6.2-KO mice was associated with depletion of subcutaneous and abdominal fat compared to WT controls (FIGS. 1E-G).

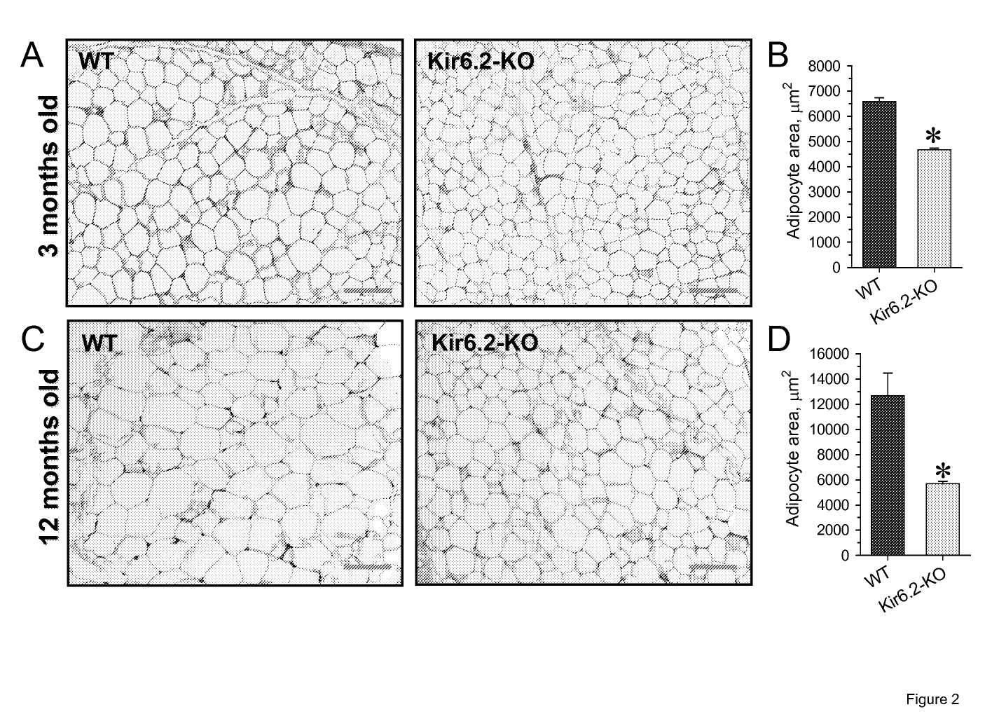

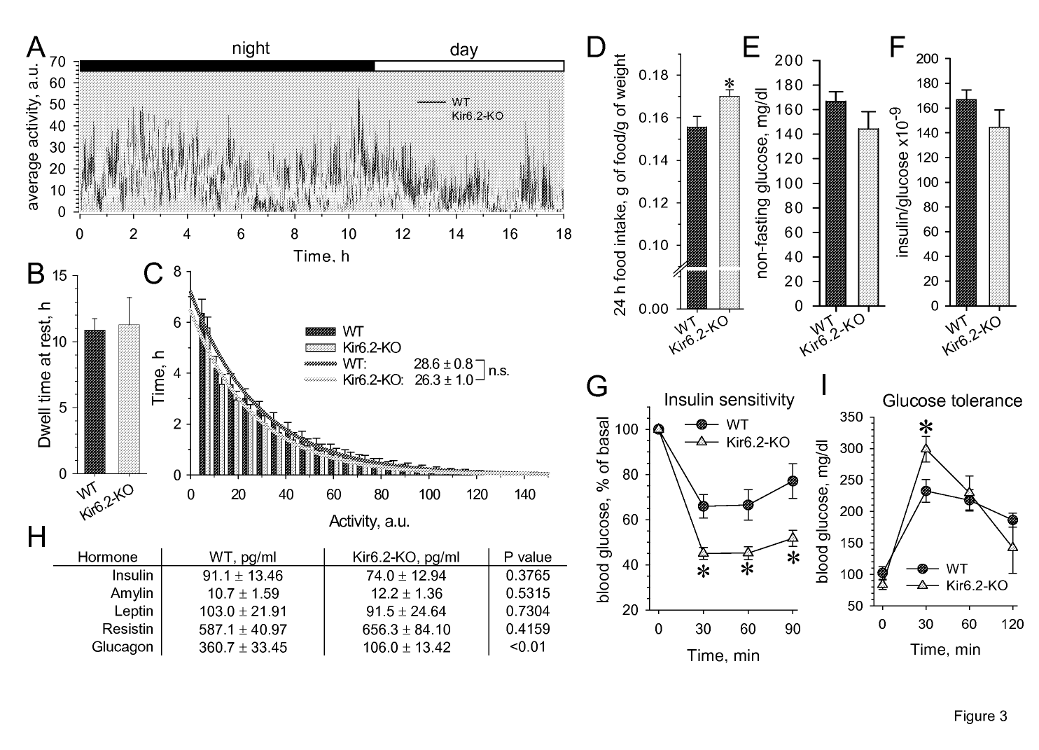

Macroscopic differences in body weight and fat distribution were preceded by reduced white adipose tissue size in Kir6.2-KO mice already at 3-months of age, as revealed on histological examination (FIGS. 2A and 2B). The development of WT mice was associated with a doubling of the average adipocyte area within 12 months, not observed in Kir6.2-KO mice (FIGS. 2C and 2D). Thus, despite equal growth with WT mice, the Kir6.2-KO phenotype was characterized by low body weight and fat storage, indicating at the whole body level mismatch between energy availability and consumption was induced by disruption of K_{ATP} channels.

### Non-Exercise Activity and Energy Input Unaffected by K_{ATP} Channel Knockout

In sedentary conditions, non-exercise activity defines variation in body weight gain. Continuous telemetry monitoring, during the day-night cycle, detected similar dynamics in the activity of sedentary WT (n=10) and Kir6.2-KO (n=10) mice (FIG. 3A). Both WT and Kir6.2-KO animals spent equal time without movement (FIG. 3B) or at any level of activity (FIG. 3C). Thus, non-exercise physical activity can be excluded as a cause of reduced body weight in the K_{ATP} channel deficient mice.

Food intake defines energy availability at the organism level. At similar serum leptin levels (FIG. 3D), Kir6.2-KO mice exhibited higher food intake than WT mice (FIG. 3E), eliminating diminished energy supply as grounds for K_{ATP} channel knockout-induced body weight change.

Glucose is a major energy equivalent at tissue levels. A similar non-fasting blood glucose concentration (FIG. 3F), marginal impairment in glucose tolerance (FIG. 3G), comparable levels of serum insulin, amylin, and resistin (FIG. 3D), equivalent glucose/insulin ratio (FIG. 3H) despite lower serum glucagon levels (FIG. 3D), and a higher insulin sensitivity (FIG. 3I) collectively ruled-out a deficit in energy equivalents at organ levels in Kir6.2-KO mice compared to WT mice. The unaffected non-exercise activity and energy input suggest that the reduced body weight caused by K_{ATP} channel deficit must be related to aggravated energy expenditure.

### Increased Energy Expenditure in K_{ATP} Channel Knockout

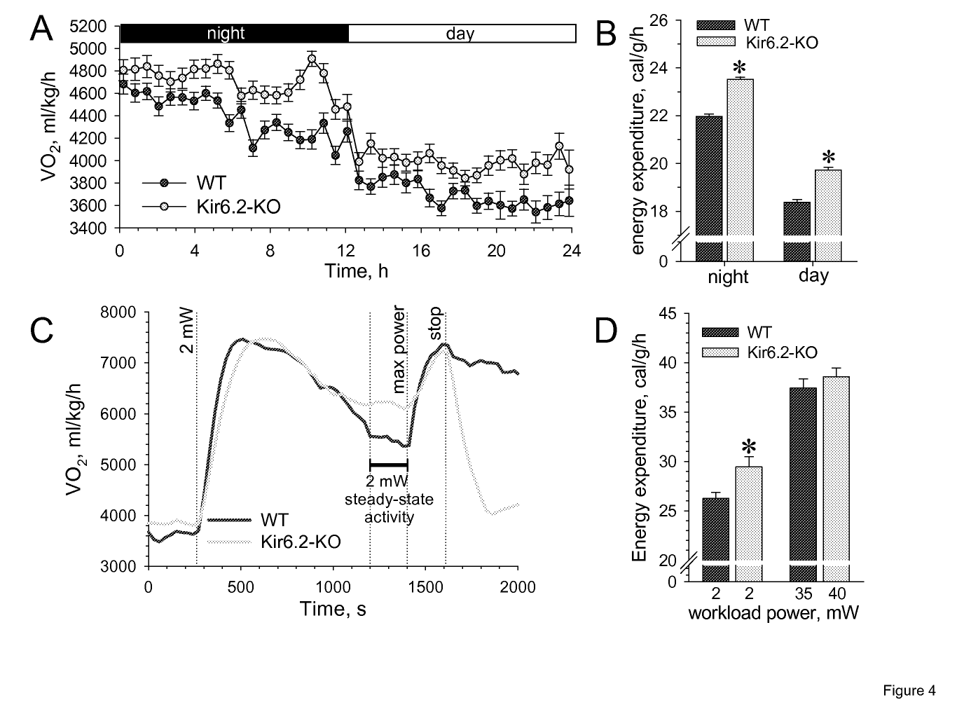

Organism energy expenditure (EE), comprised of energy required for performance of basal physiological functions and physical activity maintenance, can be accurately measured by indirect calorimetry based on rate of oxygen uptake (V O₂). To minimize the contribution of metabolically inactive adipose tissue on calculated energy expenditure, WT and Kir6.2-KO cohorts were recruited at an age point prior to divergence in body weight and gross fat distribution. Under sedentary conditions, during continuous 24-hour monitoring, Kir6.2-KO mice exhibited a higher rate of oxygen consumption throughout the 12-hour light cycles compared to age-, sex-, weight-, and body fat distribution-matched WT mice (FIG. 4A). Concomitant measurement of the rate of O₂ consumption and CO₂ production revealed a higher rate of caloric output normalized to whole body weight, reflecting a higher energy expenditure (EE=3.815·V O₂+1.232·V CO₂), during both night and day, in Kir6.2-KO mice (n=11) compared to WT mice (n=11; FIG. 4B). Thus, reduced body weight and white fat depots in 6.2 KO mice are caused by increased energy resources consumption.

### K_{ATP} Channel Dysfunction Aggravates Energetic Cost of Physical Activity

Energetically demanding physical activity is important for organism existence and survival. To evaluate energetic cost of physical activity or adaptive energy expenditure, indirect calorimetry was performed on Kir6.2-KO mice and WT mice during an exercise treadmill test. At an inclination of 5 degrees, treadmill speed was individually set according to body weight to achieve a workload 2 mJ/s (2 mW). With initiation of exercise test and despite prior training and adaptation, all animals experienced a stress-induced transient elevation of oxygen consumption ultimately reaching steady levels when oxygen consumption matched workload (FIG. 4C). At steady-state, the energetic cost of 2 mW workload was significantly higher in Kir6.2-KO mice compared to WT mice expressed either as rate of oxygen consumption or energy expenditure (FIGS. 4C and 4D). Thus, disruption of K_{ATP} channel function is associated with elevated energetic cost of physical activity.

### Aggravated Energetic Cost of Physical Activity in K_{ATP} Channel Knockout Caused by Increased Energy Consumption

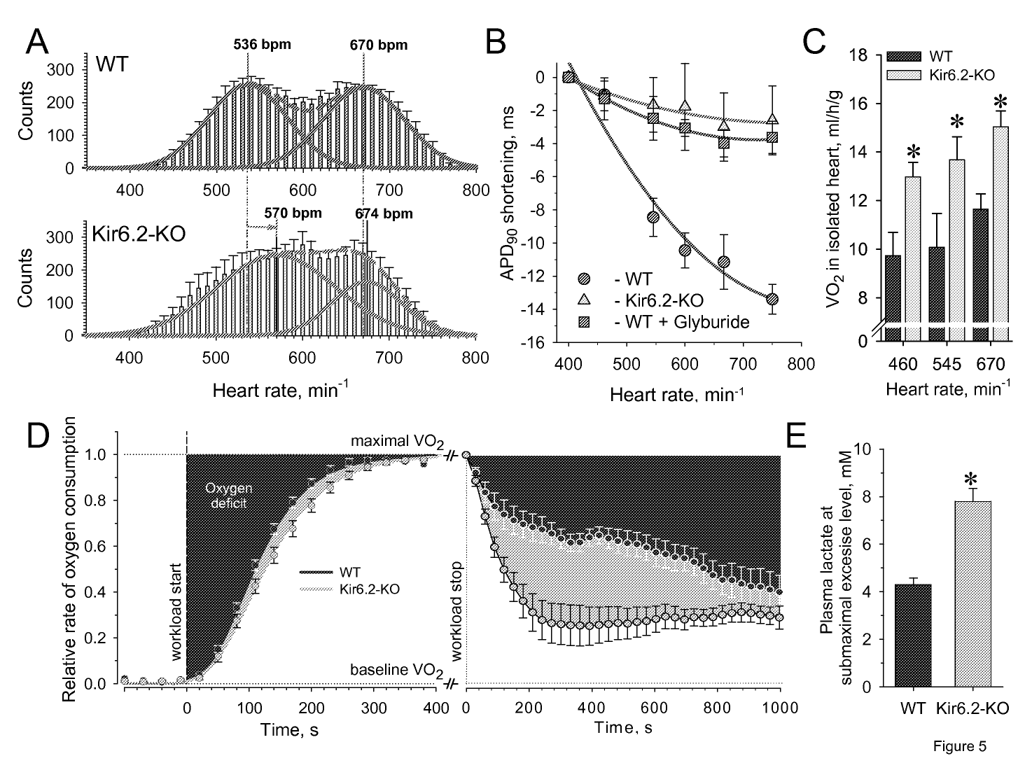

Activity-related energy expenditure largely reflects the performance of cardiac and skeletal muscle, tissues with abundant K_{ATP} channel expression. Accordingly, cardiac muscle was used as a model of striated musculature. As cardiac function and associated energy consumption depends on electrical determinants, the contribution of K_{ATP} channels in heart rate-dependent modulation of action potential duration and rate of oxygen consumption were tested. Telemetry recording of cardiac activity defined that both WT and Kir6.2-KO mice display a heart rate profile with a 400 to 800 beats per minute range (FIG. 5A). Pacing perfused hearts isolated from WT and Kir6.2-KO mice to cover the physiological heart rate range revealed defective rate-dependent modulation of action potential duration in the absence of K_{ATP} channels (FIG. 5B). While the WT mice (n=5) prominently shortened the duration of monophasic action potential duration at 90% repolarization (APD₉₀) with an increase in heart rate, hearts from Kir6.2-KO mice (n=8) exhibited a significantly reduced action potential shortening especially at high heart rates (P<0.05; FIG. 5B). The K_{ATP} channel-dependent component in rate-dependent action potential shortening was verified in hearts from WT mice following K_{ATP} -channel blockade with the sulfonylurea blocker glyburide (FIG. 5B). The inability to limit action potential duration translated into significantly higher levels of oxygen consumption in the absence of functional K_{ATP} channels (FIG. 5C). Thus, K_{ATP} channel deficit aggravates the energetic cost of physical activity through augmented energy consumption.

### Inefficient Energetics in K_{ATP} Channel Knockout Fail to Support Increased Demand of Activity Related Workload

Maximal oxygen consumption (V O₂ₘₐₓ), corresponding to the highest tolerated workload, reflects the maximal rate of energy production and the ability of the cardiovascular system to supply oxygen. Kir6.2-KO and WT mice achieved the same level of V O₂ₘₐₓ reflecting their equivalent oxidative phosphorylation potential. However, Kir6.2-KO animals reached V O₂ₘₐₓ level already at 30-35 mW (n=7), a value of workload power significantly lower than the 40-45 mW when WT mice reached this level (n=7; FIG. 4D). Thus, the increased energetic cost of physical activity in the absence of K_{ATP} channels compromises maximal workload performance due to failure to support activity-related demand.

The function of K_{ATP} channels in energy resources maintenance during physiological physical activity was analyzed. Animals were allowed to exercise on running wheels with respect to their spontaneous day and night life, intensity, and pattern. The average number of run attempts, running distance, time, and speed were calculated. This analysis revealed that in spite of the same number of exercise events, WT mice run faster, longer, and further. Thus, normal K_{ATP} channel function is involved in maintaining energy resources and normal organism performance.

Increased Energy Consumption Associated with K_{ATP} Channel Dysfunction Caused High-Fat Diet Obesity Resistance

Obesity can be a problem of energy balance, wherein adipose tissue stores can accumulate to excess levels and expenditure does not match the intake. To examine the role of K_{ATP} channels in obesity management, WT and Kir6.2-KO mice were challenged with a “western” type high-fat diet. Food intake, body weight, body mass index, and rate of the body weight gain were determined.

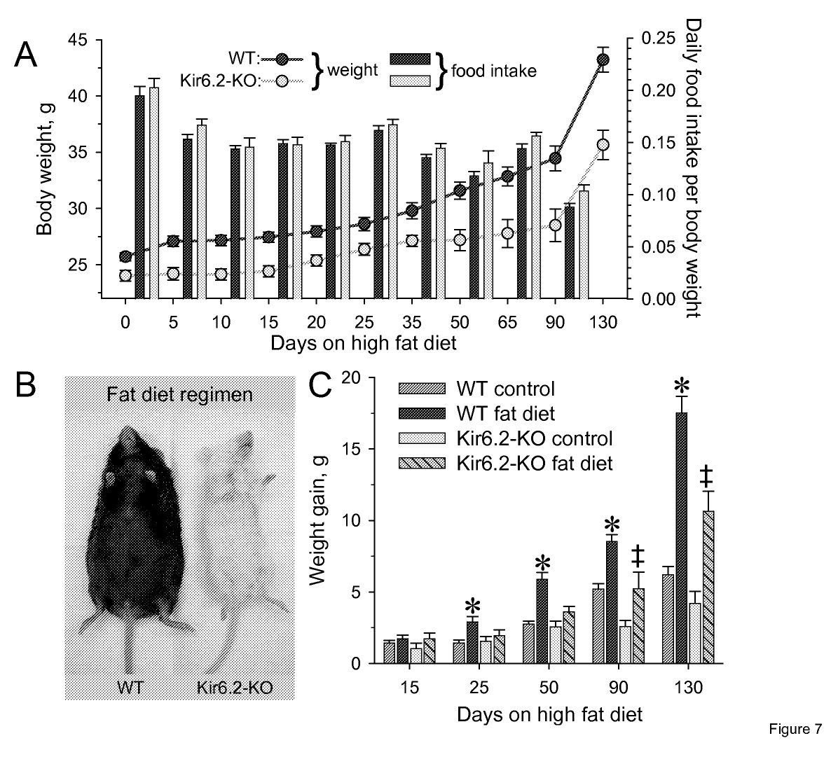

Kir6.2-KO mice exhibited significantly lower and slower body weight gain in spite on the larger food consumption (FIG. 7). Thus, K_{ATP} channel dysfunction associated EE increase provides protection from diet-induced obesity. K_{ATP} channels appear involved in physical endurance and stress adaptation. Therefore, potential targets for obesity treatment can be downstream to the K_{ATP} channels in the EE cascade regulation.

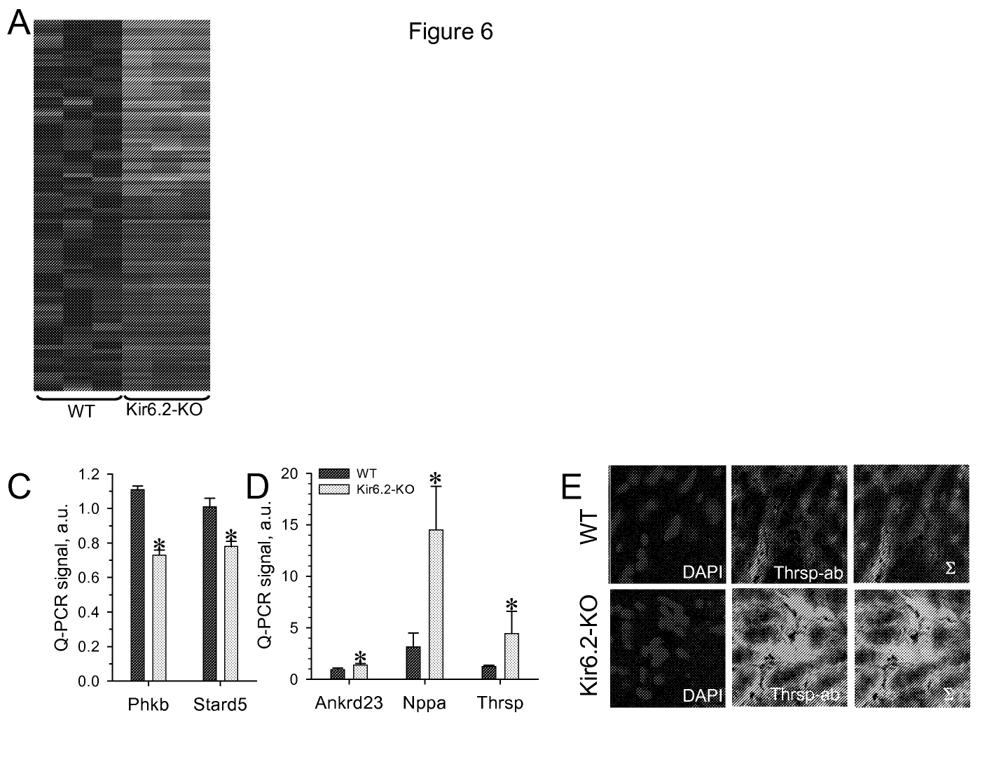

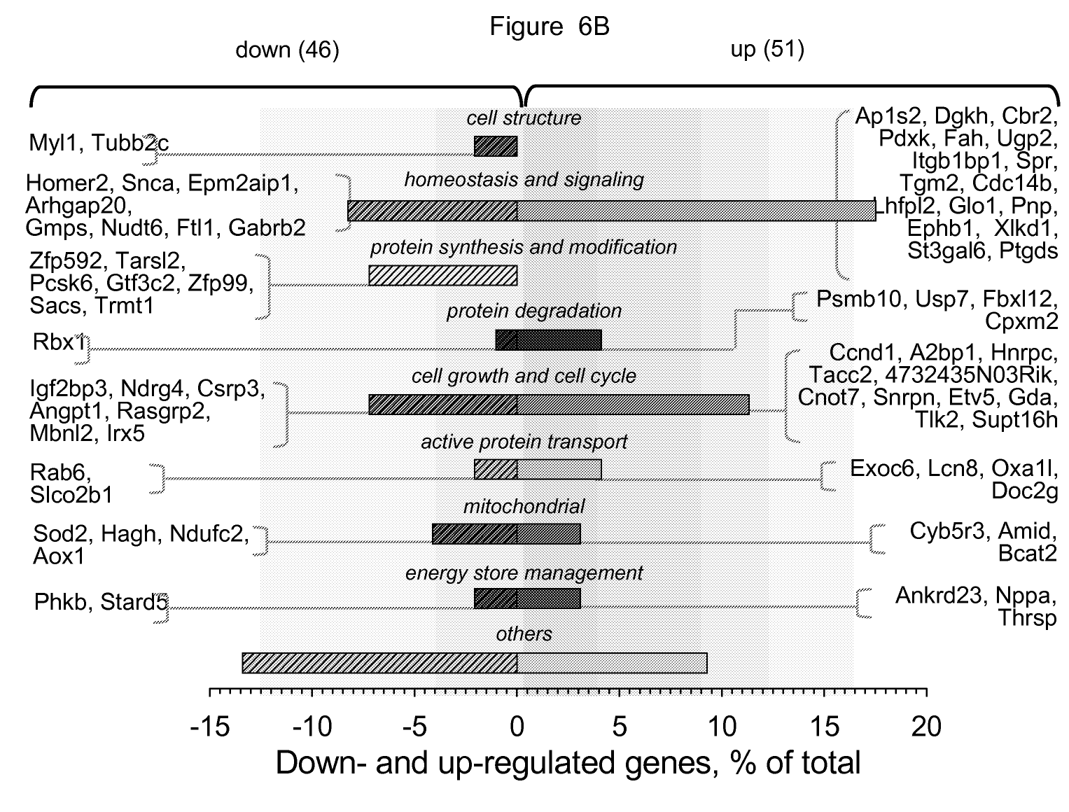

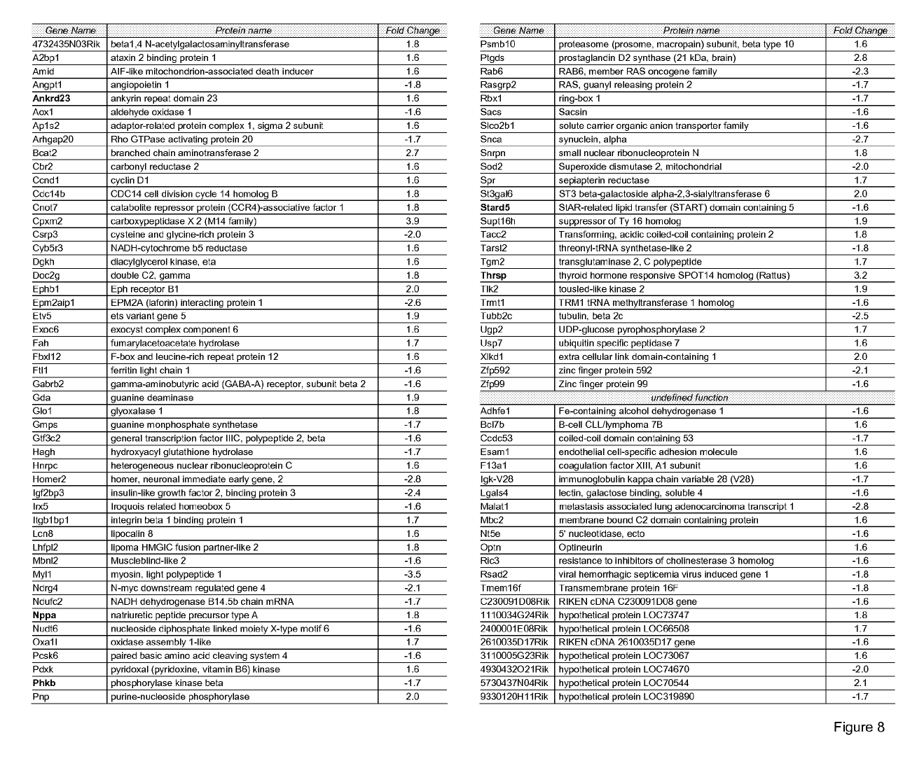

Metabolically active myocardial tissue were used to perform Affymetrix microarray gene chip analysis. This analysis revealed 97 genes which have more than 50% change in the level of mRNA expression in hearts from Kir6.2-KO mice, when compared to age- and gender-matched WT controls (FIGS. 6 and 8). These changes were verified by QT-PCR, and immunocytochemistry for Phkb, Stard5, Ankrd23, Nppa, and Thrsp (FIG. 6C, FIG. 6D and FIG. 6E).

Two of the identified proteins, thyroid hormone responsive protein and ANP, have been previously identified as regulators of lipid deposition/mobilization and were significantly up-regulated in Kir6.2-KO mice compare to WT controls. Thus, K_{ATP} channel dysfunction caused energetic inefficiency and gene reprogramming resulting in obesity resistance.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for modulating body weight in a mammal, wherein said method comprises administering, to said mammal, a composition comprising a potassium channel agonist, a potassium channel antagonist, an agent having the ability to increase potassium channel expression in said mammal, or an agent having the ability to decrease potassium channel expression in said mammal.

2. The method of claim 1, wherein said mammal is a human.

3. The method of claim 1, wherein said mammal is overweight, and said composition comprises said potassium channel antagonist.

4. The method of claim 3, wherein said composition is administered to said mammal under conditions wherein the body weight of said mammal decreases.

5. The method of claim 3, wherein said potassium channel antagonist is HMR 1883, HMR 1098, 5-hydroxydecanoate, 1[5-[2-(5-chloro-o-anisamido)ethyl]-2-methoxyphenyl]sulfonyl-3-methylthiourea, or sulfonylurea drugs.

6. The method of claim 1, wherein said mammal is overweight, and said composition comprises said agent having the ability to decrease potassium channel expression in said mammal.

7. A method for identifying a treatment agent for modulating body weight, wherein said method comprises:
(a) determining whether or not a test agent modulates potassium channel activity, wherein modulation of said potassium channel activity indicates that said test agent is a candidate agent, and
(b) administering said candidate agent to a mammal to determine whether or not said candidate agent modulates the weight of said mammal or modulates the rate of weight gain in said mammal, wherein a modulation of said weight or said rate of weight gain indicates that said candidate agent is said treatment agent.

8. The method of claim 7, wherein said step (a) comprises using an in vitro potassium channel activity assay.

9. The method of claim 7, wherein said mammal is a mouse.
